(19) United States
(12) Patent Application Publication
(10) Pub. No.: US 2020/0268979 A1
(43) Pub. Date: Aug. 27, 2020

(54) DELAY DEVICE FOR A MEDICAL ADMINISTRATION APPLIANCE

(71) Applicant: Ypsomed AG, Burgdorf (CH)

(72) Inventors: Marc Fiechter, Konolfingen (CH); Stefan Burren, Schwarzenburg (CH); Markus Tschirren, Burgdorf (CH); Markus Hunziker, Gelterkinden (CH)

(21) Appl. No.: 15/930,698

(22) Filed: May 13, 2020

Related U.S. Application Data
(63) Continuation of application No. PCT/IB2018/058902, filed on Nov. 13, 2018.

(30) Foreign Application Priority Data
Nov. 21, 2017 (CH) 01411/17

(51) Int. Cl. A61M 5/315; A61M 5/31; A61M 5/20

(52) U.S. Cl. CPC A61M 5/31511; A61M 5/3148; A61M 2005/3132; A61M 2005/2073; A61M 5/20

(57) ABSTRACT

A piston rod unit for a device for administering a medical substance delays a release of a mechanism of the device and includes an outer piston rod with a hollow space and a contact face for interacting with a stopper to discharge the substance, and an inner piston rod mounted in the outer piston rod to form a cavity at the distal end of the outer piston rod. The cavity contains a viscous fluid and a fluid displacement element located at least partially in the cavity that is operably or integrally connected to the inner piston rod. During relative movement between the fluid displacement element and the outer piston rod, fluid displacement causes movement resistance, such that mechanism release is delayed. The outer piston rod includes a distal, first piston rod part, comprising the contact face and the cavity, and a second piston rod part adjoining the first.

### RELATED APPLICATIONS

This application is a continuation of International Patent Application No. PCT/IB2018/058902, filed Nov. 12, 2018, entitled “DELAY DEVICE FOR A MEDICAL ADMINISTRATION APPLIANCE,” which in turn claims priority to Swiss Patent Application No. 01411/17, filed Nov. 21, 2017, entitled “DELAY DEVICE FOR A MEDICAL ADMINISTRATION APPLIANCE”, each of which is incorporated by reference herein, in the entirety and for all purposes.

### TECHNICAL FIELD

The present invention relates to the field of medical administration appliances for administering liquid substances, in particular insulin and hormone preparations. The invention relates to a plunger rod unit for a medical administration appliance. The plunger rod unit comprises an outer and an inner plunger rod. The inner plunger rod interacts with a fluid displacement element that is located in a cavity with a viscous fluid, so that in the case of a relative movement between the fluid displacement element and the outer plunger rod, a movement resistance is generated by displacement of the fluid, as a result of which a triggering of a mechanism of the administration appliance can be delayed.

### BACKGROUND

In medical administration appliances, it can be desirable that certain functions can be delayed. Thus, for example, in an injection process it can be necessary that, after the injection of the medical substance, such as, for example, insulin, the injection needle is not immediately pulled out again. In addition, certain medical substances, in particular viscous drugs, take a certain time in order to reach the body of the patient. For these reasons, a final process such as, for example, a needle retraction mechanism or an end signal, such as, for example, a beep, is delayed, so that the user does not terminate the administration process early and does not pull out the needle too early.

From the patent specification EP 2542280 B1, an autoinjector is known, in which the syringe is pulled back into the autoinjector after the injection. The retraction mechanism is triggered after a plunger rod has shifted the stopper of the pre-filled syringe by a certain distance. Here, the syringe retraction is not carried out immediately after the certain distance has been reached, but instead only after a certain delay. Immediately after the certain distance has been reached, due to the movement of the plunger rod, a release part is released, which starts to rotate due to the action of a pretensioned torsion spring. It is only after the release part has rotated by a specific angle that the syringe retraction is released. The rotation of the release part thus generates a delay in connection with the retraction of the syringe. In the specification, the triggering of the syringe retraction is further delayed, in that the rotation of the release part is slowed down by a viscous substance.

The patent application US 2017/0087304 A1 describes a delay device of an injector, the aim of which is to delay an acoustic or tactile end signal when a medical substance has been completely injected. The delay device comprises a plunger rod drive that comprises a tubular housing, a sleeve-shaped delay element rotatably mounted in this housing as well as a cylindrical signal generation element coaxially mounted in the delay element. The signal generation element here interacts with a plunger rod that acts on a container with the drug. At the end of an injection, a force from the plunger rod drive sets the delay element in rotation relative to said plunger rod drive and relative to the signal generation element. Between the inner surface of the housing of the plunger rod drive and the sleeve outer surface of the delay element and between the sleeve inner surface and the outer surface of the signal generation element, a grease is located, which damps the rotation of the delay element and thereby delays the end signal from the signal generation element.

WO 2016/205962 A1 discloses an additional delay element in which, at the end of an injection, a release mechanism indicating the end of the injection is delayed. For this purpose, the injector comprises an outer plunger rod with a cavity and a bore, and an inner plunger rod mounted in the bore. The inner plunger rod is connected to a rudder-like element that protrudes into the cavity. Said cavity is filled with a viscous fluid. During the injection, via a drive, the outer and the inner plunger rods are moved toward one another in the axial direction and push against a stopper of a container filled with a medical substance so that the medical substance is discharged. At the end of the injection, the inner plunger rod is detached from a guide, so that it can rotate relative to the outer plunger rod. The rotation of the inner plunger rod is here damped by the rudder-like element, since said rudder-like element experiences resistance due to the fluid in the cavity. As a result, the release mechanism is delayed.

Such known delay devices have the disadvantage that they are difficult to produce. The viscous fluid must be filled in during assembly on the assembly device for the medical administration appliance. Since the production process for the administration appliance must meet high requirements with regard to particulate and biological cleanliness, the filling of the viscous fluid is extremely difficult and expensive.

The aim of the claimed device is to provide an administration appliance with a delay device that is easy to construct and produce.

### DESCRIPTION OF THE INVENTION

The aim is achieved by the features disclosed and claimed. According to the disclosure, an outer plunger rod is designed in two parts with a distal first plunger rod part that comprises a contact face for interacting with a stopper for discharging the substance from the product container and wherein the first plunger rod part forms a cavity that can be filled with a viscous fluid, and a second plunger rod part adjoining the first plunger rod part. Preferred embodiments are the subject matter of the dependent claims.

Because the outer plunger rod is designed so as to form two parts, during the assembly, the distal first plunger rod part with the viscous fluid and the fluid displacement element can be pre-assembled spatially separate from the second plunger rod part. The first plunger rod part can be adjusted in terms of its size to the fluid displacement element. Thereby, the first plunger rod part with the viscous fluid can be pre-mounted or pre-assembled to form a compact unit that is easy to handle. By this pre-assembly, the risk of contaminating the other components of the administration appliance with the fluid can be reduced. Preferably, the first plunger rod part with the fluid displacement element and a sealing element that is present in any case is pre-mounted to form a unit that is sealed with respect to the filled fluid, so that the fluid cannot flow out of the cavity. This compact sealed unit can subsequently be brought into the assembly environment of the second plunger rod part and the remaining components of the administration appliance. This simplifies the assembly of the plunger rod unit. In order to prevent inclusion of air bubbles, the filling of the outer plunger rod with a highly viscous thick fluid may occur in a vacuum. A sealing closure of the cavity under vacuum also results in a negative pressure that additionally contributes to the sealing. This filling process is considerably more space saving and simpler to carry out with a first plunger rod part that is adjusted in terms of its size to the volume of the fluid than with a single piece outer plunger rod with which only a comparatively small volume is introduced. By the additional process step of filling and sealing under vacuum, the smallest possible units that can be separately cleaned and tested are produced before the further assembly according to the invention.

In addition, the two-part outer plunger rod according to the description enables the production of each of the two plunger rod parts from a different material. Therefore, the plunger rod unit in the area of the first plunger rod part and in the area of the second plunger rod part can have different physical properties that are particularly advantageous for the respective location. Thus, for example, the first plunger rod part can be produced from a plastic that has a special chemical resistance and is therefore particularly suitable for interacting with a viscous fluid, whereas the second plunger rod part can be produced, for example, from a plastic that is advantageous with regard to the production and the assembly or that has special sliding properties, so that the inner plunger rod can be assembled in a particularly easily movable manner. This means that the plunger rod unit can be constructed with the two-part outer plunger rod in such a manner that it meets the respective local requirements particularly well.

In addition, the scope of disclosure also covers the individual parts of the plunger rod unit that can be assembled to form the plunger rod unit. The plunger rod unit comprises an outer plunger rod with a contact face on a distal end, wherein the outer plunger rod is designed to be formed in two parts with a distal first plunger rod part that comprises the contact face, forms a cavity, and can be closed in a fluidically sealing manner by means of a gasket and a fluid displacement element, and with a second plunger rod part that can be connected to the first plunger rod part. The plunger rod unit moreover comprises an inner plunger rod that is designed in such a manner that it can be mounted in the outer plunger rod, a fluid displacement element for displacing a fluid in the cavity, and a gasket for sealing between the fluid displacement element and the first plunger rod part.

In the present description, the term “distal” refers to a direction toward the injection (or needle) end of the administration appliance. In contrast, the term “proximal” refers to a direction toward the rear end of the administration appliance, which faces the injection end. In this context, the term “drug” or “medical substance” denotes any flowable medical formulation that is suitable for the controlled administration by means of a cannula or a hollow needle, for example, a liquid, a solution, a gel or a fine suspension containing one or more medical substances. A drug can thus also be a composition with a single substance or a premixed or co-formulated composition with multiple active substances from a single container. The term drug comprises in particular medicines such as peptides (for example, insulins, insulin-containing drugs, GLP-1 containing preparations as well as derived or analogous preparations), proteins and hormones, biologically prepared or active substances, active substances based on hormones or genes, nutrition formulations, enzymes and other substances either in solid (suspended) or liquid form. The term also denotes polysaccharides, vaccines, DNA or RNA, or oligonucleotides, antibodies or parts of antibodies as well as suitable base substances, adjuvants and carrier substances.

In the present description, the term “administration appliance” is understood to mean an appliance by means of which a medical substance can be injected from a product container. The injection can occur, for example, intra-cutaneously, subcutaneously or intramuscularly. The administration appliance can accordingly be designed, for example, as a disposable injection system for single, manual or preferably automatic, administration of a preset, preferably maximum, dose from the product container. A disposable injection system is a system that is used to inject the medical substance located in a non-refillable and non-exchangeable carpoule. If the quantity of medical substance provided for the injection has been injected in one or more injection processes, the disposable injection system is exchanged. The carpoule in the disposable injection system cannot be exchanged. In contrast, in a reusable injection system, the carpoules with the medical substance are exchangeable.

According to the disclosure, a release position of the administration appliance that releases the mechanism with delay can be achieved. Such a mechanism preferably enables a function of the administration appliance that is performed at the end of an administration process. In this way, the mechanism can output, for example, an acoustic, optical or tactile end signal in order to indicate the end of the administration process to the user. An acoustic end signal can be, for example, a beep or a mechanical click sound. An optical end signal can be formed, for example, by an appearance of a display element in a window in the administration appliance, by a change in the color of an element or by a change in the shape of an element. A tactile end signal can be implemented in the form of a vibration that can be sensed by the user, an impact or a change of shape in the handle area of the administration appliance. However, the mechanism can also comprise, for example, a needle retraction device known from the prior art, which pulls the needle out of the skin after the administration process and retracts it into the administration appliance.

The term “plunger rod” is understood to mean a device that establishes a physical connection between a drive unit and a product container and by means of which a force can be transmitted by the drive unit to a stopper in the product container.

In a preferred embodiment, the plunger rod has a longitudinal shape. Preferably, it is designed essentially in the shape of a cylinder. However, the plunger rod does not necessarily have to have a circular cross section. The cross section can have, for example, a rectangular, a polygonal, or an elliptic shape. Preferably, the entire plunger rod unit can be shifted within a housing of the administration appliance relative to the housing and in the direction of the housing. Thereby, the stopper of the product container can be shifted for discharging the product from the product container in the longitudinal direction.

The outer and the inner plunger rods are two individual parts that can be produced separately and independently of one another. In the assembled state, the inner and the outer plunger rod can be connected detachably or nondetachably to one another. Likewise, the first and the second plunger rod parts are individual parts that can be produced individually and separately from one another. In the assembled state of the plunger rod unit, the first and the second plunger rod parts are connected detachably or nondetachably to one another.

The outer plunger rod has a hollow space that is formed by the outer plunger rod parts that are connected to one another. The hollow space is designed at least in sections essentially in the shape of a cylinder and has an opening on a proximal end of the second plunger rod part. Such a hollow space can also be referred to as a blind hole, wherein the production is in no way limited to drilling. Preferably, the first plunger rod part forms the closure of the hollow space. For this purpose, the first plunger rod part comprises a cavity. Said cavity is preferably subdivided into several chambers. However, in alternative embodiments, the cavity can also be designed to be cylindrical, rectangular, hemispherical or in the shape of a shell in the first plunger rod part. The second plunger rod part is preferably in the shape of a sleeve, wherein the second plunger rod part forms the cylindrical section of the hollow space of the outer plunger rod.

In a preferred embodiment, the fluid displacement element is designed as a single part that can be connected to the inner plunger rod in a detachable manner, for example, by means of a detachable snap closure or screw connection, or nondetachably, for example, by gluing or welding. This means that the fluid displacement element is not designed so as to form an integral part of the inner plunger rod. In an alternative embodiment, the fluid displacement element can be designed not as an individual part but as a protrusion, a wing or a projection on the inner plunger rod. In this case, the fluid displacement element is integrally connected in the inner plunger rod and is thus not a part that can be produced separately from the inner plunger rod.

The fluid is preferably a nonvolatile liquid and it can be, for example, a simple Newtonian liquid. The delay can be set via the viscosity of the liquid.

The plunger rod unit moreover comprises a gasket for fluidically sealing the cavity. By means of the seal, the risk of fluid flowing out of the cavity can be reduced. The gasket can be formed, for example, in the form of a sealing element such as, for example, a sealing ring, or else by a precise fitting of an outer dimension of the fluid displacement element to an inner dimension of the first plunger rod part. The gasket thus does not necessarily have to be designed as an independent component. Preferably, the gasket is a sealing element in the form of an O-ring that is arranged in a rotationally symmetrical region of the fluid displacement element.

In a preferred embodiment, the inner plunger rod is axially fixed in the longitudinal direction of the plunger rod unit relative to the outer plunger rod. This means that, when the inner plunger rod is moved by a drive unit in the longitudinal direction relative to a housing of the administration appliance in order to shift the stopper of the product container to discharge the product, the outer plunger rod, that is to say the first and the second plunger rod parts, are entrained.

Preferably, the outer plunger rod is held by a positive-locking connection on the inner plunger rod so that the outer plunger rod cannot be shifted in the longitudinal direction relative to the inner plunger rod. For this purpose, the inner plunger rod preferably has a protrusion in the radial direction, preferably in the form of a cam, a nose or a peripheral collar. This protrusion can interact with a closure surface, a ledge, or a recess in the outer plunger rod so that the inner plunger rod is mounted in such a manner that it cannot be moved in the longitudinal direction relative to the outer plunger rod. However, for this purpose, the outer plunger rod can alternatively also comprise a protrusion and the inner plunger rod can comprise a complementary element.

Preferably, the outer plunger rod is configured in such a manner that at least half the length of the inner plunger rod is accommodated in the outer plunger rod. Thereby, the plunger rod unit can be constructed compactly. In a preferred embodiment, the length of the first plunger rod corresponds essentially to the length of the fluid displacement element, so that the fluid displacement element can be largely accommodated in the first plunger rod part. Moreover, the length of the second plunger rod part corresponds essentially to the length of the inner plunger rod without fluid displacement element, so that the inner plunger rod without fluid displacement element can largely be accommodated by the first plunger rod.

Preferably, during the delay movement, the fluid displacement element can rotate about a longitudinal axis of the outer plunger rod relative to the outer plunger rod. By a rotation of the fluid displacement element relative to the outer plunger rod, the fluid located in the cavity is displaced, whereby the movement resistance and thus the desired delay are generated. In contrast to a fluid displacement element that can be shifted in the longitudinal direction of the plunger rod unit, a construction with a rotatable fluid displacement element takes up less space and the plunger rod unit can be constructed compactly.

In one embodiment, during the rotation of the fluid displacement element, the fluid is displaced in peripheral direction and in the radial direction. Alternatively, in a corresponding design of the fluid displacement element, for example, with an angled surface, the fluid can be displaced primarily in the longitudinal direction.

In one embodiment, the fluid displacement element can rotate by an angle of at least 60°, preferably at least 90°, and particularly preferably between 100° and 120°, relative to the outer plunger rod. The rotation angle is a function of the shape of the fluid displacement element and of the design of the cavity. The greater the rotation angle, the longer that the release of the mechanism can be delayed. Thus, by the selection of the shape of the fluid displacement element and of the cavity in the first plunger rod part, the delay time can be set. As mentioned above, the delay time can moreover be set by way of the viscosity of the fluid.

In an alternative embodiment, the possibility that the fluid displacement element can rotate by less than 60° exists. The delay of the release of the mechanism of the administration appliance is correspondingly shorter in this case.

Preferably, the fluid displacement element has a pin for transmitting axial forces and for centering the fluid displacement element in the cavity. Preferably, the pin is used for transmitting axial forces for the discharging of the medical substance. Here, the axial forces are transmitted by the inner plunger rod to the fluid displacement element. Said fluid displacement element further transmits the axial forces by means of the pin to the first outer plunger rod part, from which they are transmitted to the stopper of the carpoule. It has been shown that by means of this force transmission via the pin, undesired friction forces during the movement of the fluid displacement element can be kept as low as possible.

By means of the pin, a bending or shifting of the fluid displacement element due to forces generated during the displacement of the fluid can be avoided. Advantageously, the pin is designed as a cylindrical pin at the distal end of the fluid connection element and accommodated in a recess in the cavity in the first plunger rod part. During a rotation of the fluid displacement element, said fluid displacement element is centered by the pin in the cavity and the fluid displacement element is stabilized via the pin. As a result, said fluid displacement element cannot be bent and tilted by the radial forces generated during the displacement of the fluid.

Preferably, the fluid displacement element comprises at least two wings for the displacement of the fluid in the cavity. By means of the at least two wings, the viscous fluid can be displaced particularly well, so that the desired movement resistance and thus the delay can be effectively generated. Advantageously, the wings protrude in the radial direction from a central portion of the fluid displacement element. In a preferred embodiment, the at least two wings are arranged with mutual offset by 180° in the rotation direction.

Alternatively, the fluid displacement element can comprise no wings and have instead, for example, an elliptic shape in cross section, and, during the rotation of the fluid displacement element, the fluid is displaced by this shape. In addition, the fluid displacement element can also have a circular cross section, and the free volume of the cavity is present only as a thin gap, in particular as an annular gap between an inner surface of the first plunger rod part and the fluid displacement element. The movement resistance in this case is generated only by the shearing resistance within the fluid.

Advantageously, the cavity comprises a first chamber for receiving a first wing of the at least two wings and a second chamber for receiving the second wing of the at least two wings. By the subdivision of the cavity into two chambers, the volume in which the fluid can move is limited, so that an effective movement resistance is generated, when each of the wings of the fluid displacement element moves in a chamber.

Advantageously, the fluid has a kinematic viscosity between 100,000 to 500,000 centistokes. With a kinematic viscosity in this range, the fluid is sufficiently viscous so that a movement resistance can effectively be achieved in order to be able to delay the release of the mechanism correspondingly. The duration of the delay can be set via the properties of the fluid. Preferably, the fluid is a Newtonian liquid, in particular a liquid silicone formulation.

However, the fluid can also be a non-Newtonian liquid, in particular a liquid with thixotropic properties. A non-Newtonian liquid is a liquid the viscosity of which changes under load as a function of time. This means that, in a thixotropic liquid, the viscosity is reduced as a function of time. Thixotropic liquids can be highly viscous in the resting state and at the extreme behave in a manner similar to solids. A shearing load decreases the viscosity considerably as a function of time under some circumstances. Numerous thixotropic liquids are available to the person skilled in the art, in particular silica gel-containing or hyaluronic acid-containing liquids. A good alternative to thixotropic liquids consists of pseudoplastic liquids which, like thixotropic liquids, have an elevated viscosity in the resting state. In contrast to thixotropic fluids, in pseudoplastic liquids, the viscosity decreases with increasing shearing rate and not as a function of time. For example, polymer solutions or ionomers are mentioned here, which can have the desired properties. However, in another embodiment, the fluid can also be an anti-thixotropic (rheopectic) liquid, in which the viscosity increases as a function of time under mechanical stress.

In one embodiment, the fluid displacement element is designed in such a manner that an single gap exists between the fluid displacement element and an inner surface of the first plunger rod part, so that, in the case of a relative movement between fluid displacement element and outer plunger rod, the fluid is forced through the gap. The gap has an approximately constant clear width of several millimeters over its length. In an alternative embodiment, the gap can also have a width of less than one millimeter. The width of the gap can be dimensioned as a function of the desired movement resistance, in order to set the delay time. In addition, the width of the gap can be adjusted to the flow properties of the fluid.

If the fluid displacement element is rotatably mounted relative to the outer plunger rod, the gap is preferably located in the radial direction between an outer surface of the fluid displacement element, in particular between an outer surface of a wing and an inner wall of the first plunger rod part. When the fluid displacement element is moved relative to the inner wall, the fluid is displaced in the cavity and can flow in the gap from one side of the fluid displacement element to the other side. In this way, thanks to the gap, the fluid displacement element cannot jam when it moves within the cavity.

Alternatively, the possibility exists that no gap is present. In this case, the fluid is pressurized, or it can flow, for example, through an opening in the fluid displacement element from one side to the other side.

Preferably, the cavity is designed to be impermeable to fluids, so that the fluid remains in the cavity during the relative movement between the fluid displacement element and the outer plunger rod. According to the invention, the delay is achieved by displacing the viscous fluid in the cavity of the first plunger rod part. Here, the fluid preferably remains within the cavity, so that, by the movement of the fluid displacement element, the fluid can be displaced as many times as desired in the cavity, and, as a result, a movement resistance can be generated each time. If the cavity is subdivided into multiple chambers, the fluid preferably remains in these chambers during the movement of the fluid displacement element. Here, during the movement, the fluid can flow, for example, from a first chamber into a second chamber without, however, leaving these chambers.

In an alternative embodiment, the fluid can be displaced from the cavity by the movement of the fluid displacement element of the outer plunger rod. In this case, the release position can be reached with delay only once.

The invention further relates to a delay unit for the medical administration appliance. For discharging the medical substance, the administration appliance comprises a discharge element that can be moved by a drive. The delay unit comprises a container that can be introduced into the administration appliance and that is filled at least almost completely with a viscous fluid (for example, under a vacuum,) a fluid displacement element that can be moved with respect to the container in a delay direction and that can be coupled to the discharge element, and a gasket between the container and the fluid displacement element for sealing enclosure of the viscous fluid. Here, the delay unit can be mounted in the medical administration appliance in such a manner that, toward the end of a discharge movement of the discharge element, the fluid displacement element and the coupled discharge element move in the delay direction into a release position.

In one embodiment, the movable discharge element is implemented as an inner plunger rod that is located in an outer plunger rod. The outer plunger rod is preferably designed in two parts with a distal first plunger rod part that is designed as a container and with a second plunger rod part adjoining the first plunger rod part. In the release position of the fluid displacement element and of the discharge element, as mentioned further above, a mechanism for generating an end signal or a mechanism for retracting a needle is triggered.

Furthermore, the invention relates to an administration appliance for administering a medical substance, comprising a signal generation mechanism for generating a signal and a plunger rod unit according to the claims, wherein, by means of the plunger rod unit, at the end of a discharge process, the generation of a signal with the signal generation mechanism can be delayed.

The signal generation mechanism can generate a signal that indicates to the user that the administration process has ended, that is to say that the desired dose of the medical substance has been injected and that the cannula or needle can be retracted. As mentioned above, the generated signal can be, for example, an acoustic, optical or a tactile signal. The acoustic signal can be, for example, a click sound, a beep or a ring. Thus, for the generation of such an acoustic signal, the signal generation mechanism can contain a spring for moving two parts in order to generate a click sound, or an electronics unit with a loudspeaker for generating a tone. The optical signal can be formed, for example, by a change in color, a change in shape or by an appearance of a display element. The tactile signal can be in the form of a vibration that can be sensed by the user or in the form of a single impact. In this case, the signal generation mechanism can contain, for example, a mechanical oscillating circuit with an electromagnet and a spring. Furthermore, the possibility exists that the signal represents a combination of an acoustic and a tactile signal. In this case, the signal can comprise, for example, a vibration with an additional click sound.

Preferably, the plunger rod unit delays a rotation of the inner plunger rod relative to the outer plunger rods. When the inner plunger rod has reached an end position after the delay, the signal generation mechanism is triggered.

Moreover, the disclosure relates to an administration appliance for administering a medical substance, comprising a housing, an insertion device arranged in the housing for inserting a needle or a cannula, a retraction mechanism for retracting the needle or cannula into the housing or into a drug container, and a plunger rod unit according to any one of the claims. Here, at the end of an administration process, the retraction of the needle or cannula by the retraction mechanism can be delayed.

At the end of the administration process, the plunger rod unit delays the retraction, so that the cannula or a needle is not pulled out directly after the discharge of the medical substance. Thereby, the medical substance can be injected correctly, and the risk of under-dosage is reduced. After the delay, the plunger rod unit triggers the retraction mechanism. This means that the retraction mechanism pulls the needle or cannula back into the housing or into the drug container of the administration appliance, so that the needle or cannula is pulled out of the insertion site.

Preferably, the plunger rod unit delays a rotation of the inner plunger rod relative to the outer plunger rods. When the inner plunger rod has reached a release position after the delay, the needle or cannula is pulled out of the insertion site in the proximal direction by the retraction mechanism.

In a preferred embodiment, the administration appliance is an injector. Examples of an injector are autoinjectors, disposable injectors, and reusable injectors.

The term “injector” is understood to mean a device by means of which the injection needle is removed from the tissue of the patient after administration of the medical substance. Thus, in the case of an injector, in contrast to an infusion system, the injection needle does not remain in the patient permanently or for a longer duration of several hours.

In a preferred embodiment, the injector is designed as a disposable injector. A disposable injector is an injector that is used to inject the medical substance located in a non-refillable or non-exchangeable carpoule. After the quantity of the medical substance provided for the injection has been injected in one or more injection processes, the disposable injector is replaced. The carpoule in the disposable injector cannot be exchanged. In contrast, in a disposable reusable injector, the carpoule with the medical substance can be exchanged. When the plunger rod unit according to the invention is inserted in a disposable injector, the fluid displacement element is preferably moved in each injection process in a direction other than the direction of the preceding injection process. In another embodiment, the fluid displacement element can be moved in each injection process by a further distance in the disposable injector.

Alternatively, the administration appliance can be an infusion system in which, after an administration process with the plunger rod unit, an end signal is delayed. An infusion system can be, for example, a patch pump or an insulin pump.

Furthermore, the invention relates to a method for producing a plunger rod unit for an administration appliance for administering a medical substance, comprising the steps

a. providing a two-part outer plunger rod with a distal first plunger rod part that comprises a cavity and with a second plunger rod part that is designed in the shape of a sleeve;

b. filling a viscous fluid into the cavity;

c. introducing a fluid displacement element into the cavity, so that at least a portion of the fluid displacement element is immersed in the viscous fluid, and mounting a sealing element or closure of a gasket between the fluid displacement element and an inner wall in the cavity, so that cavity is fluidically sealed;

d. connecting the first plunger rod part to the second plunger rod part, so that the first plunger rod part cannot move relative to the second plunger rod part in the rotation direction and in the longitudinal direction and wherein the fluid displacement element is located within the first plunger rod part and within the second plunger rod part.

If the fluid displacement element is designed as a single separate part and not so as to be integrally connected on the inner plunger rod, the method in addition comprises the steps of

e. coaxial introduction of an inner plunger rod into the outer plunger rod;

f. connecting the inner plunger rod to a distal end of the inner plunger rod with the fluid displacement element, so that the inner plunger rod cannot be moved relative to the fluid displacement element in the rotation direction and in the longitudinal direction.

Here, the rotation direction is the direction about the longitudinal axis of the outer plunger rod, and the longitudinal direction is the direction along the longitudinal axis of the outer plunger rod.

In a preferred embodiment, the filling of the fluid into the cavity occurs under vacuum or negative pressure. During a filling under vacuum or negative pressure, inclusions of air bubbles can be avoided or reduced.

### FIGURES

FIG. 1 shows an administration appliance with a plunger rod unit according to the invention in a section view, wherein the section extends through the longitudinal axis of the administration appliance;

FIG. 2 shows an enlargement of the section view from FIG. 1 in a distal region of the plunger rod unit;

FIG. 3 shows an exploded view of the plunger rod unit; and

FIG. 4 shows a perspective view of the first plunger rod part of the plunger rod unit.

### DESCRIPTION

FIG. 1 shows a section view of an administration appliance designed in the form of an autoinjector 1. The section here extends along a central longitudinal axis L of the autoinjector 1.

The autoinjector 1 comprises a sleeve-shaped longitudinal housing 2 with the longitudinal axis L. In this housing 2, a product container 3 with the liquid medical substance, a product container holder referred to as syringe holder 4, a plunger rod unit 10 with an outer plunger rod 30 and with an inner plunger rod 20, and a drive unit 6 are arranged. On a distal end of the autoinjector 1, said autoinjector comprises in its delivery state a pull-off cap 5a that has to be removed before the use of the autoinjector 1.

Below, the most important components of the autoinjector 1 are briefly described. A detailed description of the autoinjector is disclosed in the patent application WO 2016/205962 A1 (incorporated by this reference). In contrast to the present invention, the autoinjector described in WO 2016/205962 A1 comprises no plunger rod unit 10 with a two-part outer plunger rod, but has instead an integrally connected outer plunger rod.

The drive unit 6 of the autoinjector 1 comprises a coil spring 7 which, in the delivery state of the autoinjector 1, is pretensioned with sufficient energy to be able to discharge the medical substance contained in the product container 3 by shifting a stopper 3a out of the product container by means of a discharge stroke. The drive unit 6 moreover comprises a threaded rod 9 oriented in the longitudinal direction L, which is in threaded engagement with the inner plunger rod 20 of the plunger rod unit 10. The inner plunger rod 20 is secured in the longitudinal direction relative to the outer plunger rod 30, so that, in the case of a rotation of the threaded rod 9, the inner plunger rod 20 and the outer plunger rod 30 move in the longitudinal direction toward the distal end relative to the housing 2. The outer plunger rod 30 includes a distal first plunger rod part 30a and a sleeve-shaped second plunger rod part 30b proximally adjoining the first plunger rod part 30a. The first plunger rod part 30a and the second plunger rod part 30b are connected to one another in such a way that they cannot be moved relative to one another in the rotational and longitudinal directions.

The first plunger rod part 30a comprises on its distal end a contact face 35. With said contact face, the first plunger rod part 30a interacts with a stopper 3a of the product container 3 in order to discharge the medical substance from the product container 3. The syringe holder 1 cannot be moved relative to the housing and is engaged with said housing. The product container 3 is arranged relative to the housing 2 in such a way that the needle tip of an insertion needle 5 protrudes distally over the distal end of the housing 2 but is covered before and after the injection by a needle protection sleeve. On its proximal end, the housing 2 comprises a closure cap 8, which is connected by positive-locking connection to the housing 2 in a rotationally and axially fixed manner and which forms the proximal end of the autoinjector 1.

For the discharge of the medical substance from the product container, the drive unit 6 drives the threaded rod that is in threaded connection with the inner plunger rod 20. Thereby, an axial movement is generated, resulting in the plunger rod unit 10 being able to shift the stopper in the carpoule in the axial direction. The axial forces occurring in the process are transmitted by the inner plunger rod 20 to the fluid displacement element 14. Via the centering tip (pin) 19 of the fluid displacement element 14, the axial forces are further transmitted to the first plunger rod part 30a, which transmits the forces via its contact face 35 to the stopper 3a.

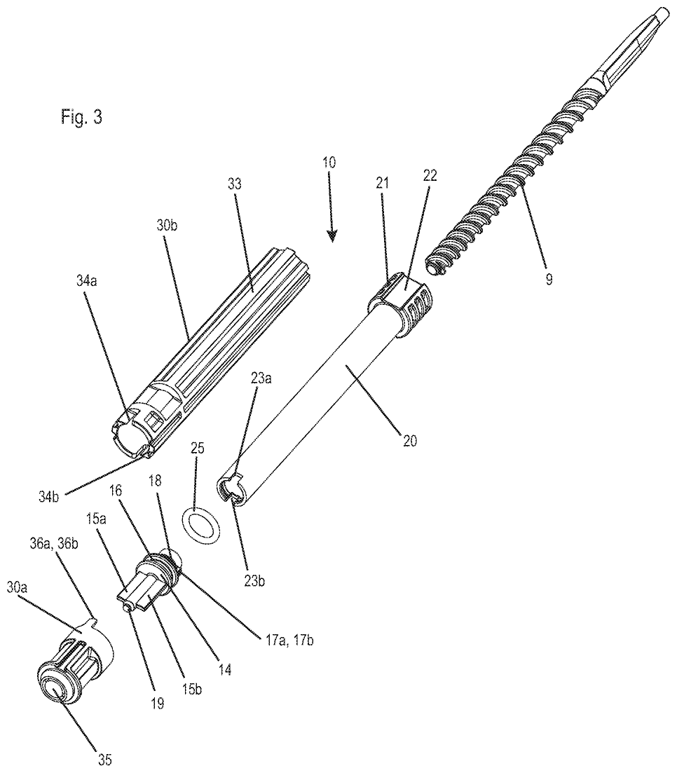

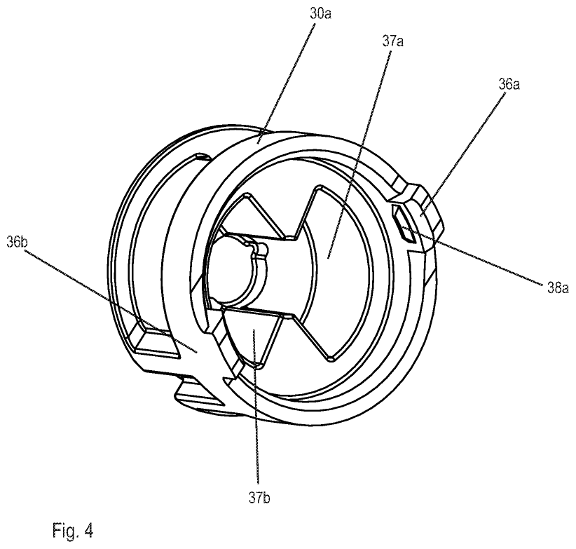

Below, the plunger rod unit 10 according to the invention is explained in detail. FIG. 3 shows an exploded view of the plunger rod unit 10 according to the disclosure. It comprises the two-part outer plunger rod 30, the inner plunger rod 20, a fluid displacement element 14 connected to the inner plunger rod 20, and an O-ring 25 that is arranged between the fluid displacement element 14 and the inner plunger rod 20.

As mentioned above, the outer plunger rod 30 comprises a distal first plunger rod part 30a and a second plunger rod part 30b proximally adjoining the first plunger rod part 30a. The first plunger rod part 30a essentially has a cylindrical shape and comprises a cavity.

FIG. 4 shows an enlarged perspective view of the first plunger rod part 30a. In this enlarged representation, one can see that two chambers 37a, 37b are formed in the cavity. The chambers 37a, 37b each occupy a partial volume of a cylinder and they each have a cross section and a footprint in the shape of a circle segment.

The second plunger rod part 30b is designed to be in the shape of a sleeve and essentially has the same outer diameter as the first plunger rod part 30a. The second plunger rod part 30b is non-rotatably mounted, guided longitudinally, in a mechanism holder 40. For this purpose, on an outer surface of the second plunger rod part 30b, multiple guiding elements 33 are arranged that are designed to be longitudinal and oriented in the longitudinal direction and that extend over almost the entire length of the second plunger rod part 30b. Complementary thereto, on an inner surface of the mechanism holder 40, longitudinal guides are arranged, into which the guiding elements 33 engage. The mechanism holder 40 cannot be shifted relative to the housing 2 along the longitudinal axis L and is arranged in a rotationally fixed manner.

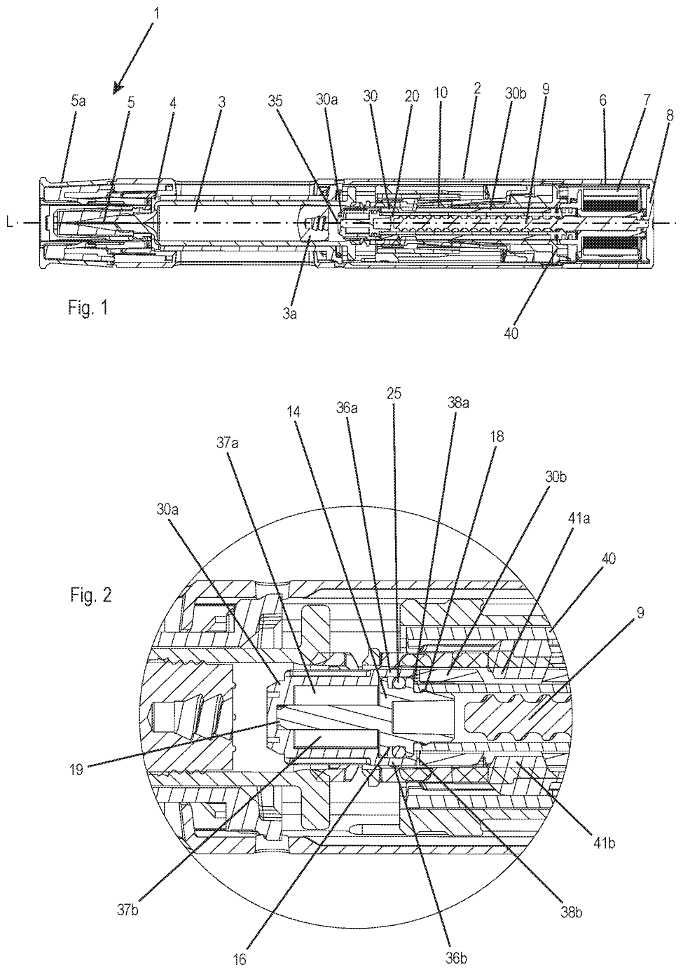

As one can see in FIGS. 1 and 2, the inner plunger rod 20 is mounted in the second plunger rod part 30b of the outer plunger rod 30. The inner plunger rod 20 is designed to be in the shape of a sleeve and has a slightly thickened proximal end compared to the rest of the sleeve body. Said proximal end has approximately the same diameter as the outer plunger rod 30 and is arranged outside of the outer plunger rod 30. Due to the thickened end, an annular structure 21 is formed on the proximal end of the inner plunger rod 20. This structure 21 is interrupted by two grooves 22. The grooves 22 have two functions. The first function is to ensure the rotation lock between the inner plunger rod 20 and the mechanism holder 40, and the second function is the release of engagement elements 41a, 41b after completion of the product discharge. On its distal end, the inner plunger rod 20 comprises two mutually facing recesses 23a, 23b. A cam 17a, 17b of the fluid displacement element 14 engages in each of these recesses 23a, 23b, so that the fluid displacement element 14 is connected in a rotationally fixed manner to the inner plunger rod 20.

In a proximal region, the fluid displacement element 14 comprises a cylindrical section that protrudes into the inner space of the inner plunger rod 20, as can be seen in FIG. 2. In this section of the fluid displacement element 14, the two cams 17a, 17b are arranged. Between the cams 17a, 17b, a peripheral collar 18 extends, which, in the assembled state, engages by positive-locking connection in a corresponding depression in the inner space of the inner plunger rod 20. The fluid displacement element 14 is thereby held in the longitudinal direction by a positive-locking connection in the inner plunger rod 20. Between this cylindrical section and a distal region, the fluid displacement element 14 comprises a central region with a peripheral groove 16. In this groove 16, an O-ring 25 is accommodated. The distal region of the fluid displacement element 14 comprises two wings 15a, 15b that radially protrude in the form of two rectangular elements, as can be seen in FIG. 3. On its distal end, the fluid displacement element 14 comprises a cylindrical centering tip (pin) 19 on the longitudinal axis L. This centering tip 19 supports the fluid displacement element 14 in the radial direction and thus ensures that, even under exposure to radial forces, the fluid displacement element 14 is always oriented on the longitudinal axis L.

In the assembled state, the distal and the central regions of the fluid displacement element 14 are located within the cavity of the first plunger rod part 30a of the outer plunger rod 30, while the proximal region (cylindrical section) of the fluid displacement element 14 is accommodated in the inner plunger rod 20. This arrangement can be seen best in the enlarged view in FIG. 2. There, it can be seen that the O-ring 25 in the groove 16 seals the cavity. The two wings 15a, 15b of the fluid displacement element 14 are each located in one of the chambers 37a, 37b of the first plunger rod part 30a. Due to the shape of the chambers 37a, 37b, the fluid displacement element 14 can rotate the wings 15a, 15b through an angle of at least 90° relative to the first plunger rod part 30a. In an embodiment, the fluid displacement element 14 can rotate by an angle of 110° relative to the first plunger rod part 30a. The chambers 37a, 37b are almost completely filled with a viscous fluid, in particular with an oily, viscous, gel-like or pasty liquid. Preferably, a silicone liquid having a kinematic viscosity between 100,000 and 500,000 centistokes is used. The first plunger rod part 30a has two arms 36a, 36b projecting in the longitudinal direction and engaging in corresponding recesses 34a, 34b in the second plunger rod part 30b. The first plunger rod part 30a is as a result secured in a rotationally fixed manner with respect to the second plunger rod part 30b. Furthermore, on their ends, the arms 36a, 36b each comprise a radially inward pointing cam 38a, 38b, each engaging behind a shoulder on the fluid displacement element 14, so that the first plunger rod part 30a cannot be moved in the longitudinal direction relative to the second plunger rod part 30b. Thus, the first and the second plunger rod parts 30a, 30b are held in such a manner that they cannot be moved relative to one another in the rotation direction and in the longitudinal direction.

On the other hand, the inner plunger rod 20 together with the fluid displacement element 14 can rotate relative to the outer plunger rod 30 (and thus relative to the first plunger rod part 30a and the second plunger rod part 30b) when the inner plunger rod 20 is released from the mechanism holder 40, as explained below.

At the beginning and during the discharge of the product from the product container, a rotation lock exists between outer plunger rod 30, inner plunger rod 20, and mechanism holder 40. At the end of a discharge, when the inner plunger rod 20 is shifted from proximal by approximately the discharge stroke toward distal, the rotation lock of the inner plunger rod 20 is released, because in this state the grooves 22 are no longer guided by ribs of the mechanism holder 40. A torque introduced by the drive unit 6 via the threaded rod 9 then induces a rotation of the inner plunger rod 20 relative to the mechanism holder 40 and the outer plunger rod 30. Because the fluid displacement element 14 is connected in a rotationally fixed manner to the inner plunger rod 20, in this state the fluid displacement element 14 rotates with the inner plunger rod 20. The resistance generated by the viscous fluid opposes the rotation. This resistance delays the rotation of the inner plunger rod 20 without, however, preventing it, since, during a rotation of the fluid displacement element 14, the fluid can flow in a gap between a radial outer surface of the wings 15a, 15b and an inner surface of the chamber from one side of the wings 15a, 15b to the other side.

During the rotation of the inner plunger rod 20, an axial force exerted by the threaded rod 9 on the plunger rod unit 10 continues to be applied and ensures that the stopper of the product container 3 is fully pressed against a distal abutment in the product container 3 during the rotation.

If the inner plunger rod 20 has rotated far enough so that the grooves 22 have the same orientation as the engagement elements 41a, 41b, a holding element is released for an axial movement in the proximal direction. The pretensioned holding element as a result springs in the proximal direction up to an abutment and generates a click sound that indicates the end of the injection process to the user.

The properties of the plunger rod unit can be influenced by the shape of the cavity, the properties of the fluid as well as by additional elements such as wing-like, rudder-like or dough-hook-like structures on inner surfaces of the fluid displacement element and by an inner or outer plunger rod.

### LIST OF REFERENCE NUMERALS

1 Autoinjector
2 Housing
3 Product container
3a Stopper
4 Syringe holder
5 Needle
5a Pull-off cap
6 Drive unit
7 Spring
8 Closure cap
9 Threaded rod
10 Plunger rod unit
14 Fluid displacement element
15a, 15b Wing
16 Groove
17a, 17b Cam
18 Collar
19 Centering tip
20 Inner plunger rod
21 Structure
22 Groove
23a, 23b Recesses
25 Sealing element
30 Outer plunger rod
30a First plunger rod part
30b Second plunger rod part
33 Guiding elements
34a, 34b Recesses
35 Contact face
36a, 36b Arms
37a, 37b Chambers
38a, 38b Cams
40 Mechanism holder
41a, 41b Engagement elements

1. A plunger rod unit for an administration appliance for administering a medical substance, wherein the plunger rod unit delays a release of a mechanism of the administration appliance, the plunger rod unit comprising:
a. an outer plunger rod with a contact face on a distal end of the outer plunger rod for interacting with a stopper, in order to discharge the substance from a product container,
b. an inner plunger rod mounted in the outer plunger rod in such a way that a cavity is formed at the distal end of the outer plunger rod, said cavity being at least partially filled with a viscous fluid,
c. a fluid displacement element that is located at least partially in the cavity and that is operably or integrally connected to the inner plunger rod and wherein the fluid displacement element is moved relative to the outer plunger rod in a delay movement,
d. wherein, in a relative movement between the fluid displacement element and the outer plunger rod by displacement of the fluid, a movement resistance is generated, so that the fluid displacement element is delayed in reaching a release position that releases the mechanism of the administration appliance, and
e. wherein the outer plunger rod comprises a distal first plunger rod part that comprises the contact face and forms the cavity for the fluid displacement element, and a second plunger rod part adjoining the first plunger rod part.

2. The plunger rod unit according to claim 1, wherein the inner plunger rod is axially fixed relative to the outer plunger rod in the longitudinal direction of the plunger rod unit.

3. The plunger rod unit according to claim 1, wherein the outer plunger rod is configured such that at least half of the length of the inner plunger rod is accommodated in the outer plunger rod.

4. The plunger rod unit according to claim 1, wherein in the delay movement, the fluid displacement element rotates about a longitudinal axis of the outer plunger rod relative to the outer plunger rod.

5. The plunger rod unit according to claim 4, wherein the fluid displacement element rotates by an angle of at least 60° relative to the outer plunger rod.

6. The plunger rod unit according to claim 1, wherein the fluid displacement element comprises a pin for transmitting axial forces and for centering the fluid displacement element in the cavity.

7. The plunger rod unit according to claim 1, wherein the fluid displacement element comprises at least two wings for displacing the fluid in the cavity.

8. The plunger rod unit according to claim 7, wherein the cavity has a first chamber for accommodating a first wing of the at least two wings and a second chamber for accommodating the second wing of the at least two wings.

9. The plunger rod unit according to claim 1, wherein the fluid has a kinematic viscosity between 100,000 and 500,000 centistokes.

10. The plunger rod unit according to claim 1, wherein the fluid displacement element is configured with a gap between the fluid displacement element and an inner surface of the first plunger rod part so that, in the relative movement between fluid displacement element and outer plunger rod, the fluid flows through the gap.

11. The plunger rod unit according to claim 1, wherein the cavity is configured so that the fluid remains in the cavity during the relative movement between the fluid displacement element and outer plunger rod.

12. The plunger rod unit according to claim 1, wherein the first plunger rod part and the second plunger rod part have different physical properties.

13. The plunger rod unit according to claim 1, wherein the delayed release of a mechanism of the administration appliance is a release of one or more of a signal generation mechanism, a retraction mechanism for retracting a needle or cannula, or a mechanism that enables a function of the administration appliance performed at an end of an administration.

14. The plunger rod unit according to claim 1, wherein the first plunger part is fluidically sealed by a sealing means cooperating with the fluid displacement element.

15. An administration appliance for administering a medical substance, comprising a signal generation mechanism for generating a signal, and a plunger rod unit according to claim 1, wherein, at the end of an administration process, the plunger rod unit delays generation of a signal by the signal generation mechanism.

16. An administration appliance for administering a medical substance, comprising a housing, an insertion mechanism arranged in the housing for inserting a needle or a cannula, a retraction mechanism for retracting the needle or cannula into the housing or into a drug container, and a plunger rod unit according to claim 1, wherein at the end of an administration process, the plunger rod unit delays retraction of the needle or cannula with the retraction mechanism.

17. A method for producing a plunger rod unit for an administration appliance for administrating a medical substance, comprising the steps of
a. providing a two-part outer plunger rod with a distal first plunger rod part that comprises a cavity and with a second plunger rod part configured as a sleeve;
b. filling a viscous fluid into the cavity;
c. introducing a fluid displacement element into the cavity, so that at least a portion of the fluid displacement element is immersed in the viscous fluid, and mounting a sealing element between the fluid displacement element and an inner wall in the cavity, so that the cavity is fluidically sealed;
d. connecting the first plunger rod part to the second plunger rod part, so that the first plunger rod part cannot move relative to the second plunger rod part rotatably or longitudinally, and wherein the fluid displacement element is located within the first plunger rod part, or within the second plunger rod part, or within both.

18. The method according to claim 17, wherein the step of filling of the fluid into the cavity occurs under vacuum or negative pressure.

19. The method according to claim 17, wherein the fluid displacement element comprises a pin for transmitting axial forces and further comprising centering the fluid displacement element in the cavity using the pin.

20. The method according to claim 17, further comprising performing a step of cleaning or testing the plunger rod unit after performing the steps a.-d. and separately from its further assembly into the administration appliance.
